(19) United States
(12) Patent Application Publication
(10) Pub. No.: US 2019/0014794 A1
(43) Pub. Date: Jan. 17, 2019

(54) PREPARATION METHOD FOR FERMENTED CORN GLUTEN

(71) Applicant: CJ CHEILJEDANG CORPORATION, Seoul (KR)

(72) Inventors: Sang Hyun SEO, Seoul (KR); Seong Jun CHO, Seoul (KR); Kyung Il KANG, Incheon (KR); Min Ju PARK, Seoul (KR); Tae Joo YANG, Seoul (KR); Seung Won PARK, Gyeonggi-do (KR)

(73) Assignee: CJ CHEILJEDANG CORPORATION, Seoul (KR)

(21) Appl. No.: 16/134,572

(22) Filed: Sep. 18, 2018

Related U.S. Application Data
(63) Continuation of application No. 14/356,870, filed on May 7, 2014.

(30) Foreign Application Priority Data
Nov. 8, 2011 (KR) 10-2011-0116043

(51) Int. Cl. A23K 10/12; C12N 1/20; C12P 21/06; A23K 20/147

(52) U.S. Cl. CPC A23K 10/12; A23K 20/147; C12P 21/06; C12N 1/20

(57) ABSTRACT

The present invention relates to a method of preparation for fermented corn gluten to improve the quality of corn gluten, which is a vegetable protein source, to a fermented corn gluten prepared by the method, and to a feed additive including the same. The present invention provides high-quality fermented corn gluten whose characteristics are improvement as a protein feed by inoculation of corn gluten, which has a high protein content but is minimally used due to low digestibility, with a Bacillus sp. strain and solid-culturing the Bacillus sp. strain, and a method of preparation therefor.

### CROSS REFERENCE TO RELATED APPLICATIONS

This application is a continuation of U.S. application Ser. No. 14/356,870, which is the National Stage of International Application No. PCT/KR2012/009417, filed Nov. 8, 2012, which claims priority to Korean Patent Application No. 10-2011-0116043, filed Nov. 8, 2011. These applications are incorporated herein by reference in their entireties.

### TECHNICAL FIELD

The present invention relates to a method of preparation for fermented corn gluten to improve the quality of corn gluten, which is a vegetable protein source, to the fermented corn gluten prepared by the method, and to a feed additive including the same.

### BACKGROUND ART

As diseases such as bovine spongiform encephalopathy (BSE), which are fatal to human beings, are proven to be due to animal protein components added to a feed, there is a rapid trend toward replacing the animal protein added to the feed with vegetable proteins.

A vegetable protein source includes corn gluten, and includes 8% of moisture, 60.4% of crude proteins, 7.7% of crude fibers, 2% of crude fats, and 6% of a crude ash, as measured for corn gluten in general component analysis. Especially, the proteins are present at a high content of up to 65%, which indicates that the proteins are present at the highest content in the vegetable source usable as a feed. Meanwhile, in an animal protein source for feeds, a fish meal having the highest protein content includes approximately 60 to 65% of proteins.

However, the vegetable proteins generally show lower digestibility than the animal proteins, and have a poor composition of essential amino acids, and low content of some vitamins, mineral matters and unknown growth factors (UGFs). Further, since corn gluten predominantly includes modified insoluble proteins formed during a preparation process, the corn gluten shows poorer digestibility than other vegetable proteins such as a soybean meal, thereby limiting the amount of a feed used.

Therefore, there is a demand for development of a novel processing method which is inexpensive and effective and can process a large amount of corn gluten, thereby improving the qualities of corn gluten, that is, digestibility of a protein fraction in order to use the corn gluten as a high-quality high-protein feed.

In studies on corn gluten, much research has been conducted to enhance commercial availability of corn gluten using a method for treating corn gluten with an enzyme or acid, which includes a method for preparing a corn gluten hydrolysate and a corn gluten hydrolysate prepared by the method (Korean Unexamined Patent Application Publication No. 2009-0121253); a method for preparing a peptide using gluten from corn and wheat (Korean Unexamined Patent Application Publication No. 1996-0022556) and a method for preparing natural condiments containing a high concentration of glutamic acid and various amino acids (Korean Unexamined Patent Application Publication No. 2009-0076428). However, such research has a problem in that since methods predominantly including a process such as acid hydrolysis or enzymatic degradation are used to prepare condiments, they can be used for foods but cannot be used for feeds due to high production costs. Therefore, there is no research currently conducted to enhance commercial availability of corn gluten in feeds.

### DISCLOSURE

### Technical Problem

The present inventors have tried to construct a production system for improving the commercial availability of corn gluten through improvement of corn gluten, which is a protein feed, and found that it is possible to prepare high-quality fermented corn gluten having enhanced digestion rate and efficiency in use as a feed due to the conversion of proteins into low molecular weight molecules, in addition to having increased protein content due to solid-fermenting the corn gluten with a Bacillus sp. strain, thereby completing the present invention.

### Technical Solution

An object of the present invention is to provide a method of preparation for fermented corn gluten including (a) inoculating moisture-containing corn gluten with a Bacillus sp. strain; and (b) solid-culturing the Bacillus sp. strain with which the corn gluten is inoculated to obtain fermented corn gluten.

Another object of the present invention is to provide fermented corn gluten prepared by the above method, which includes low molecular weight proteins.

Still another object of the present invention is to provide a feed additive including the fermented corn gluten.

### Advantageous Effects

(a) The present invention provides high-quality fermented corn gluten having improved characteristics for use as a protein feed by inoculating corn gluten, which has a high protein content but is minimally used due to low digestibility, with a Bacillus sp. strain, and solid-culturing the Bacillus sp. strain, and a method of preparation therefor.

(b) Specifically, the fermented corn gluten according to present invention shows excellent applicability and value since the fermented corn gluten has a protein content 15% higher than a similar type of fermented soybean meal known in the related art. Since conventional fermented soybean meal has a protein content of approximately 50 to 55%, the conventional fermented soybean meals do not show superior commercial availability in spite of their high qualities due to the middle positioning between a fish meal having a high protein content (60 to 65%) and a soybean meal having low protein content (45%). However, the fermented corn gluten of the present invention is considered to show very excellent commercial availability and value since the fermented corn gluten has a protein content substantially similar to the fish meal having high protein content (60 to 65%).

(c) Corn gluten for condiment matters (foods) prepared through conventional acid or enzyme treatment cannot be used for feeds due to the complexity in a process and burden of expenses, but the fermented corn gluten of the present invention can be useful in enhancing commercial availability of corn gluten as a feed since the fermented corn gluten can be produced with low production costs.

(d) Also, the fermented corn gluten prepared according to the present invention can be useful in maximizing a digestive effect upon feed intake since a Bacillus sp. strain effective as a probiotic remains in a final product in the form of spores, thereby causing an increase in value.

(e) As described above, the fermented corn gluten of the present invention can be useful in improving digestibility due to the presence of low molecular weight proteins and the like, and can be widely used as a high-quality vegetable protein feed since the fermented corn gluten has a protein content substantially similar to that of fish meal, which is a high protein product.

### DESCRIPTION OF DRAWINGS

These and other features, aspects, and advantages of preferred embodiments of the present invention will be more fully described in the following detailed description, with reference to the accompanying drawings. In the drawings:

FIG. 1 is a flowchart showing a method of preparation for fermented corn gluten according to an embodiment of the present invention;

FIG. 2 shows the SDS-PAGE results of corn gluten; and

FIG. 3 shows the degradability of proteins according to fermentation conditions for corn gluten.

(1) Corn gluten (control)

(2) 50% moisture added to corn gluten+Lactobacillus plantarum (cultured at 30° C. for 48 hours)

(3) 70% moisture added to corn gluten+Bacillus subtilis (cultured at 50° C. for 48 hours)

(4) 40% moisture added to corn gluten+B. subtilis (cultured at 37° C. for 24 hours)

(5) 50% moisture added to corn gluten+B. subtilis (cultured at 37° C. for 24 hours)

(6) 50% moisture added to corn gluten+B. subtilis (cultured at 40° C. for 24 hours)

### BEST MODE

In order to solve the above problems of the prior art, one aspect of the present invention is to provide a method of preparation for fermented corn gluten, which includes:
(a) inoculating moisture-containing corn gluten with a Bacillus sp. strain; and
(b) solid-culturing the Bacillus sp. strain with which the corn gluten is inoculated to obtain fermented corn gluten.

The respective steps of the method of preparation for fermented corn gluten according to the present invention will be described in further detail, as follows.

(a) Inoculating Moisture-Containing Corn Gluten with a Bacillus sp. Strain

It is preferred that corn gluten used in the present invention is the same kind of corn gluten produced in the same area so that the fermented corn gluten final product can maintain the same qualities. However, a difference in the quality of a raw material such as corn gluten does not affect fermentation itself. Corn gluten is a yellow powder obtained by separating and drying proteins existing in the corn, and has been increasingly commercially available as a feed.

The type of moisture-containing corn gluten used in the present invention may be used without limitation as long as it contains moisture. For example, corn gluten pretreated in advance to contain moisture may be obtained (or purchased) for use, or moisture-containing corn gluten prepared by adding moisture to the resulting corn gluten may be used herein. In this case, the addition of moisture may be performed by directly spraying a predetermined amount of water on corn gluten or mixing a predetermined amount of water with corn gluten in order to adjust the amount of moisture.

Preferably, the moisture content of the moisture-containing corn gluten may be in a range of 30 to 70% (v/w), and, more preferably in a range of 40 to 60% (v/w). When the moisture content is less than 30%, fermentation of the Bacillus sp. strain may be prolonged due to the low moisture content, and it is especially undesirable for the moisture content to reach a level of 20%, in which the Bacillus sp. strain cannot grow after final fermentation as moisture evaporates during the fermentation. When the moisture content is greater than 70%, high costs may be required during a drying process, and corn gluten may be lumped due to the small particle size, thereby causing uneven fermentation.

Preferably, the temperature of water added to the corn gluten may be in a range of room temperature to 100° C., and, more preferably in a range of 15° C. to 100° C.

According to one exemplary embodiment of the present invention, the solid-fermenting efficiency of the corn gluten by means of heat treatment was confirmed. As a result, it was confirmed that the corn gluten could be sufficiently fermented without performing an initial steaming (heat treatment) process on the corn gluten. However, since heat treatment may be performed to some extent so as to reduce contamination by various germs when the amount of the inoculated strain is decreased for the purpose of reducing prime costs, corn gluten heat-treated after addition of moisture may also be used in the present invention.

When the heat-treated corn gluten is used, corn gluten which has been heat-treated at a temperature of 50 to 120° C. for 5 to 30 minutes after addition of moisture may be preferably used herein.

According to the method of the present invention, the moisture-containing corn gluten is inoculated with the Bacillus sp. strain.

When the moisture-containing corn gluten is inoculated with the Bacillus sp. strain, the Bacillus sp. strain may be directly inoculated in a culture broth, or may be inoculated as uniformly as possible after proper dilution with sterilized water.

The amount of the inoculated Bacillus sp. strain may be in a range of 10⁷ to 10⁹ cfu/g. In this case, the amount of the inoculated Bacillus sp. strain may be preferably in a range of 10⁶ to 10⁹ cfu/g since the heat-treated corn gluten shows a sterilization effect when the heat-treated corn gluten is inoculated with the Bacillus sp. strain. When the amount of the inoculated Bacillus sp. strain is small, monetary value of yields may be low due to long fermentation time, and the culture broth may be contaminated with various germs. When the Bacillus sp. strain is inoculated at a concentration of greater than 10⁹ cfu/g, production costs may increase due to complicated conditions required for production of the inoculated Bacillus sp. strain and various medium compositions, thereby making it difficult to use the strain to prepare a feed.

Preferably, the Bacillus sp. strain may be an apathogenic Bacillus sp. strain. More preferably, the apathogenic Bacillus sp. strain may be a Bacillus sp. strain selected from the group consisting of B. subtilis, B. licheniformis, B. toyoi, B. coagulans, and B. polyfermenticus.

(b) Solid-Culturing the Bacillus sp. Strain with which the Corn Gluten is Inoculated to Obtain Fermented Corn Gluten.

One of the characteristics of the present invention is to enhance digestion by solid-culturing a Bacillus sp. strain with corn gluten to convert proteins in the corn gluten into low molecular weight molecules. In the prior art, a protein as a source of feed has been minimally used due to low digestibility of corn gluten, and while a method for processing corn gluten by means of conventional acid hydrolysis or enzymatic degradation can be used to prepare foods (condiments) due to the high production costs, but has a problem in that it can be used to prepare feeds. In this circumstance, the present invention provides a method of preparation for corn gluten including proteins converted into low molecular weight molecules through the solid-fermenting using the Bacillus sp. strain, thus significantly expanding its commercial availability as a corn gluten feed which is a large source of protein.

In the present invention, the term “solid-culturing (fermentation)” means that microorganisms are cultured using corn gluten remaining after extracting most of starch and gemmules from the corn and separating the corn bran.

Preferably, the solid-culturing may be performed at a temperature of 30 to 45° C., more preferably 35 to 40° C., and the most preferably 37° C.

The fermentation may be performed using a fermenter used to prepare typical soy sauces and fermented soybean meals, or may be performed using a drum-type fermenting machine, but the present invention is not limited thereto. For example, when corn gluten has a high moisture content, the fermentation may be performed by a method using a liquid culture tank, etc.

(c) Drying and Grinding the Fermented Corn Gluten

Preferably, the method according to the present invention may further include (c) drying and grinding the fermented corn gluten after Step (b). When the solid-fermenting is performed using a thermohygrostat or a fermenter, moisture remaining after the fermentation is present at a very high content of 20 to 50% (v/v). Therefore, a final process of reducing the moisture content is required.

The drying and grinding may be performed using various methods known in the related art. For example, when the drying is performed at an extremely high temperature, the proteins in the final product may be further deformed, thereby exerting a negative influence on a digestive effect. Preferably, a hammer mill may be used in a grinding method.

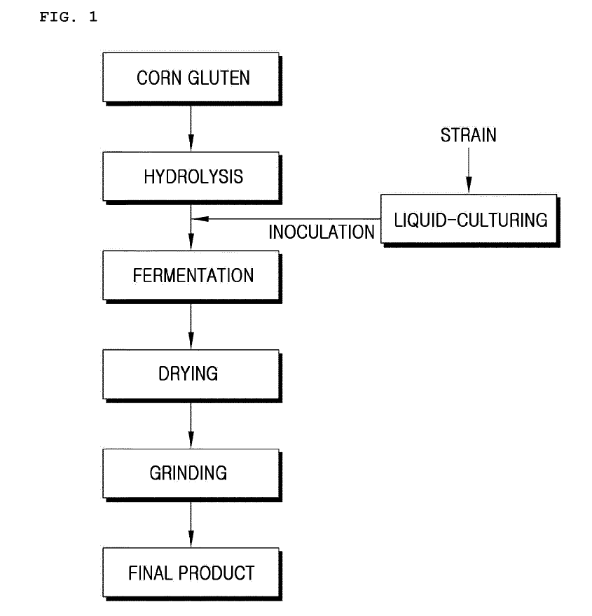

The flowchart of the method of preparation for fermented corn gluten according to one exemplary embodiment of the present invention is shown in FIG. 1.

According to another aspect, the present invention provides fermented corn gluten including low molecular weight proteins prepared by the method of preparation according to the present invention.

In connection with the fermented corn gluten, description of the same contents as described in the method of preparation for fermented corn gluten is omitted for clarity.

In the present invention, the term “low molecular weight proteins” refers to proteins having a lower molecular weight than corn gluten proteins which are not fermented by decomposing corn proteins (i.e., zein proteins) included in the corn gluten with a proteolytic enzyme through solid-fermenting. The fermented corn gluten according to the present invention is characterized in that it includes low molecular weight proteins as described above.

Preferably, the fermented corn gluten may have a protein content of 62 to 65% (w/w). Such protein content is equivalent to a content of a fish meal which is a high-quality animal protein supply source, and is greater than or equal to a protein content (48 to 55% (w/w)) of a fermented soybean meal which is a vegetable protein supply source.

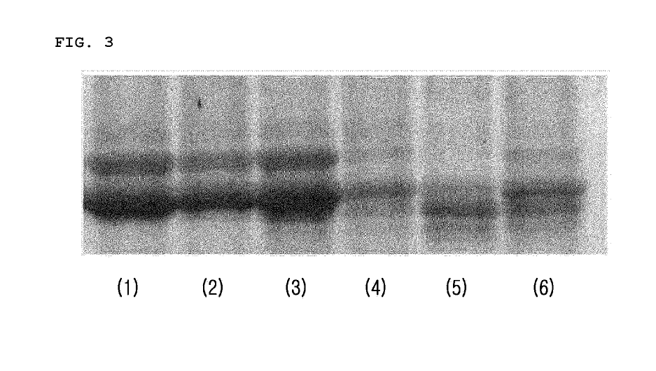

According to one exemplary embodiment of the present invention, the moisture-containing corn gluten was solid-cultured with B. subtilis. As a result, it was confirmed that the proteins in the corn gluten were degradable into low molecular weight molecules, unlike solid-culturing using the Lactobacillus sp. strain (FIG. 3). Also, it was confirmed that the protein content of the fermented corn gluten prepared by the method according to the present invention increased compared with before fermentation (Table 3). As described above, the present invention provides the fermented corn gluten, which includes low molecular weight proteins and has enhanced protein content, by solid-fermenting corn gluten with the Bacillus sp. strain, thereby significantly improving commercial availability of corn gluten as a high-quality vegetable protein source capable of replacing animal proteins.

According to still another aspect, the present invention provides a feed additive including the fermented corn gluten according to the present invention.

In the present invention, the term “feed additive” refers to a substance added to a feed so as to improve productivity of a target organism and cause health promotion.

The feed additive may be prepared in various forms known in the related art, and may be used alone or in combination with conventional feed additives known in the related art.

The feed additive may be added to the feed at a proper compositional ratio as a high-content vegetable protein supply source capable of replacing animal proteins. In this case, the compositional ratio of the feed additive may be easily determined by those skilled in the art.

The feed additive according to the present invention may be added to a feed for animals such as a chicken, a pig, a monkey, a dog, a cat, a rabbit, a cow, a sheep and a goat, but the present invention is not limited thereto. In particular, the feed additive according to the present invention may have effects of supplying a large amount of vegetable proteins and improving a digestion since the feed additive contains the fermented corn gluten.

### MODE FOR INVENTION

Hereinafter, preferred embodiments of the present invention will be described in detail referring to the accompanying drawings. Therefore, it should be understood that the description proposed herein is merely a preferable example for the purpose of illustration only, and is not intended to limit the scope of the invention, so it should be understood that other equivalents and modifications could be made thereto without departing from the scope of the invention.

### Example 1: Effect of Heat Treatment on Solid-Fermentation of Corn Gluten

The same content of moisture was added to corn gluten, and the corn gluten was then heat-treated at 60 to 120° C., and cooled to an optimum fermentation temperature of 37° C. Thereafter, the corn gluten was inoculated with B. subtilis at the same density of inoculated strains with respect to each experimental group.

TABLE 1
Solid-fermentation of corn gluten according to heat treatment

| | | No. of | No. of strain |
|---|---|---|---|
| Moisture content | | inoculated | after 24 hours |
| after moisture | Heat | strain | fermentation |
| addition | treatment | (cfu/g) | (cfu/g) |
| 50% | None | 1.5 × 10⁷ | 1.2 × 10⁹ |
| 50% | 60° C., 10 | 1.5 × 10⁷ | 3.0 × 10⁹ |
| | minutes | | |
| 50% | 60° C., 30 | 1.5 × 10⁷ | 2.2 × 10⁹ |
| | minutes | | |
| 50% | 80° C., 10 | 1.5 × 10⁷ | 2.5 × 10⁹ |
| | minutes | | |
| 50% | 80° C., 30 | 1.5 × 10⁷ | 2.8 × 10⁹ |
| | minutes | | |
| 50% | 100° C., 10 | 1.5 × 10⁷ | 3.0 × 10⁹ |
| | minutes | | |
| 50% | 100° C., 30 | 1.5 × 10⁷ | 3.6 × 10⁹ |
| | minutes | | |
| 50% | 120° C., 10 | 1.5 × 10⁷ | 3.1 × 10⁹ |
| | minutes | | |
| 50% | 120° C., 30 | 1.5 × 10⁷ | 3.2 × 10⁹ |
| | minutes | | |

As listed in Table 1, it could be seen that the inoculated strains grew regardless of the heat treatment. From the above results, it was confirmed that the corn gluten was sufficiently fermented without performing an initial steaming (heat treatment) process.

### Example 2: Confirmation of Fermentation Level of Corn Gluten According to Initial Content of Added Moisture

To determine a fermentation level of corn gluten according to an initial content of moisture, corn gluten whose moisture content after moisture addition were adjusted to 30%, 40%, 50%, 60%, 70% and 80% was inoculated with each of the Bacillus sp. and Lactobacillus sp. strains, fermented for 24 hours, and counted. The results are listed in the following Table 2.

TABLE 2
Fermentation level of corn gluten according
to initial content of added moisture

| | | No. of strain |
|---|---|---|
| Moisture content | | after 24 hours |
| after moisture | | fermentation |
| addition | Strains | (cfu/g) |
| 30% | L. plantarum | 8.2 × 10⁶ |
| 30% | B. subtilis | 1.2 × 10⁷ |
| 40% | L. plantarum | 7.0 × 10⁶ |
| 40% | B. subtilis | 1.5 × 10⁹ |
| 50% | L. plantarum | 2.8 × 10⁷ |
| 50% | B. subtilis | 3.0 × 10⁹ |
| 60% | L. plantarum | 3.6 × 10⁷ |
| 60% | B. subtilis | 3.1 × 10⁹ |
| 70% | L. plantarum | 3.6 × 10⁸ |
| 70% | B. subtilis | 3.0 × 10⁹ |
| 80% | Uniform fermentation is | — |
| | impossible due to severe | |
| | lumping | |

As a result, it was revealed that the Bacillus sp. strain (1.2×10⁷ cfu/g) slightly grew and the Lactobacillus sp. strain (8.2×10⁶ cfu/g) did not grow when the corn gluten had a moisture content of 30%, compared to the initial number (1.0×10⁷ cfu/g) of the inoculated strains. Also, it could be seen that the Bacillus sp. strain grew rapidly at a moisture content of 40% or more, which indicated that the minimum moisture content was greater than or equal to 30% when the Bacillus sp. strain was cultured with corn gluten. The Lactobacillus sp. strain was cultured more slowly than the Bacillus sp. strain, and hardly grew, especially at a moisture content of 40% or less. From these results, it could be seen that the Bacillus sp. strain growing rapidly in the corn gluten is more suitable for preparation of a final product than the Lactobacillus sp. strain.

### Example 3: Confirmation of Protein Degradability According to Fermentation Conditions of Corn Gluten

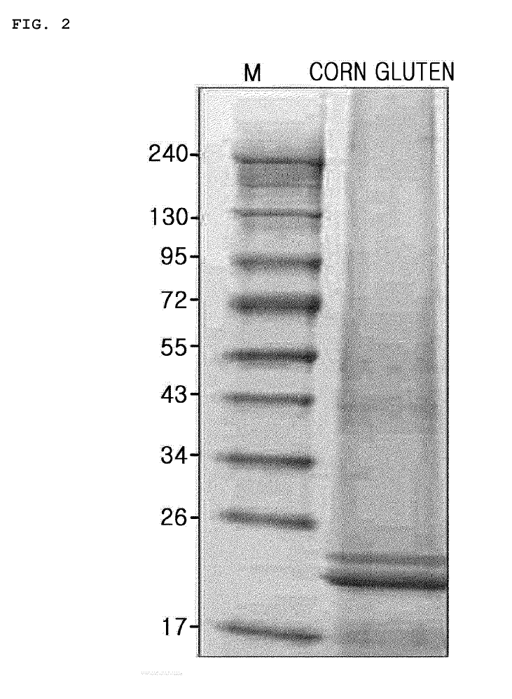

Kinds of proteins in the corn gluten were confirmed through SDS-PAGE. The results are shown in FIG. 2. From the results, it could be seen that the corn gluten was simply composed to two proteins, as shown in FIG. 2.

When the main proteins of the corn gluten shown in FIG. 2 were converted into low molecular weight molecules through fermentation, a digestive effect of the final product was improved, which indicated degradation of the proteins in the corn gluten under actual fermentation conditions. The decomposition results of the constituent proteins of corn gluten after the corn gluten having a moisture content of 50% was inoculated with L. plantarum after the moisture addition and cultured at 30° C. for 48 hours (Lane 2), the decomposition results of the constituent proteins of corn gluten after the corn gluten having a moisture content of 70% was inoculated with B. subtilis after the moisture addition and cultured at 50° C. for 48 hours (Lane 3), the decomposition results of the constituent proteins of corn gluten after the corn gluten having a moisture content of 40% was inoculated with B. subtilis after the moisture addition and cultured at 37° C. for 24 hours (Lane 4), and the decomposition results of the constituent proteins of corn gluten after the corn gluten having a moisture content of 50% was inoculated with B. subtilis after the moisture addition and cultured at 37° C. and 40° C. for 24 hours (Lanes 5 and 6) are shown in FIG. 3.

As a result, it could be seen that the two major proteins, zein proteins, of the corn gluten were not easily decomposed when the corn gluten was cultured with L. plantarum (i.e., Lactobacillus sp. strain) (Lane 2), but the decomposition rate of the proteins was high in the case of B. subtilis (Lanes 4 to 6), as shown in FIG. 3. Meanwhile, in the case of the third Bacillus experimental group, it was revealed that a fermentation level was low, which indicated that the corn gluten was cultured with the Bacillus sp. strain at a temperature of 50° C. which was higher than the optimum temperature at which the Bacillus sp. strain was able to grow. From these results, it could be seen that the optimum results were obtained when the fermentation was performed at a temperature of up to 45° C. under production conditions for the final product.

### Example 4: Confirmation of Protein Content after Fermentation of Corn Gluten

Corn gluten was fermented in the same manner as in Example 3, and the protein content of the fermented corn gluten was measured. The experimental method was performed in the same manner as in Example 3, an experiment for measuring a protein content was performed using a Kjeldahl method, and FOSS Kjeltec 8400 was used as a measuring machine. In this regard, the experimental results are listed in Table 3.

TABLE 3

| | | Protein |
|---|---|---|
| source and | | content (based |
| moisture | Strain and culture | on moisture |
| addition ratio | conditions | correction of 7%) |
| Corn gluten | Control (not cultured) | 62% |
| (control) | | |
| corn gluten and | L. plantarum (cultured at | 62% |
| 50% moisture | 30° C. for 48 hours) | |
| added | | |
| corn gluten and | B. subtilis (cultured at | 63% |
| 70% moisture | 50° C. for 48 hours) | |
| added | | |
| corn gluten and | B. subtilis (cultured at | 64% |
| 40% moisture | 37° C. for 24 hours) | |
| added | | |
| corn gluten and | B. subtilis (cultured at | 65% |
| 50% moisture | 37° C. for 24 hours) | |
| added | | |
| corn gluten and | B. subtilis (cultured at | 63% |
| 50% moisture | 40° C. for 24 hours) | |
| added | | |

As a result, it was confirmed that the protein content of the corn gluten after the fermentation was in a range of 62 to 65%, which was slightly higher than the protein content (approximately 62%) of the corn gluten before fermentation. The protein content of the fermented corn gluten was substantially similar to that of the fish meal which was a high-quality animal protein product. These results showed that the fermented corn gluten prepared by the method according to the present invention was effectively used as a high-quality vegetable protein source capable of replacing an animal protein source.

1. A method of preparation for fermented corn gluten with an increased protein content and improved digestibility compared to before fermentation, comprising:
(a) inoculating moisture-containing corn gluten with a Bacillus sp. strain; and
(b) solid-culturing the Bacillus sp. strain with which the corn gluten is inoculated to obtain fermented corn gluten,
wherein the corn gluten to which the moisture is added has a moisture content of 40 to 70% (v/w), and
wherein an amount of the Bacillus sp. strain for inoculating is 10⁶ to 10⁹ cfu/g.

2. The method of preparation of claim 1, wherein the corn gluten to which the moisture is added is heat-treated at 50 to 120° C. for 5 to 30 minutes after addition of the moisture.

3. The method of preparation of claim 1, wherein the Bacillus sp. strain is an apathogenic Bacillus sp. strain.

4. The method of preparation of claim 4, wherein the apathogenic Bacillus sp. strain is selected from the group consisting of Bacillus subtilis, Bacillus licheniformis, B. toyoi, B. coagulans, and B. polyfermenticus.

5. The method of preparation of claim 1, wherein the solid-culturing is performed at a temperature of 30 to 45° C.

6. Fermented corn gluten prepared by the method defined in claim 1, comprising a low molecular weight protein.

7. A feed additive comprising the fermented corn gluten defined in claim 7.
